(19) United States
(12) Patent Application Publication
(10) Pub. No.: US 2009/0281161 A1
(43) Pub. Date: Nov. 12, 2009

(54) Organic Compounds

(76) Inventors: Juergen Klaus Maibaum, Weil-Haltingen (DE); Edwige Liliane Jeanne Lorthiois, Niffer (FR)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/304,425

(22) PCT Filed: Jun. 11, 2007
(86) PCT No.: PCT/EP2007/005131

§ 371 Date: Dec. 11, 2008

(30) Foreign Application Priority Data
Jun. 13, 2006 (GB) 0611696.6

(51) Int. Cl. A61K 31/4025; A61K 31/40; C07D 207/14; C07D 405/12

(52) U.S. Cl. 514/422; 514/426; 548/557; 548/517

(57) ABSTRACT

Novel 3,4-di-, 3,3,4-di-, 3,4,4,-tri- and 3,3,4,4-tetra-substituted pyrrolidine compounds, these compounds for use in the diagnostic and therapeutic treatment of a warm-blooded animal, especially for the treatment of a disease (=disorder) that depends on inappropriate activity of renin; the use of a compound of that class for the preparation of a pharmaceutical formulation for the treatment of a disease that depends on inappropriate activity of renin; the use of a compound of that class in the treatment of a disease that depends on inappropriate activity of renin; pharmaceutical formulations comprising a said substituted pyrrolidine compound, and/or a method of treatment comprising administering a said substituted pyrrolidine compound, a method for the manufacture of said substituted pyrrolidine compounds, and novel intermediates and partial steps for their synthesis are described. The substituted pyrrolidine compounds are especially of the formula (I) wherein the substituents are as described in the specification.

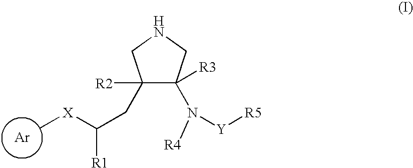

### ORGANIC COMPOUNDS

The invention relates to (3,4-di-, 3,3,4-tri, 3,4,4-tri- or 3,3,4,4-tetra-) substituted pyrrolidine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising said substituted pyrrolidine compound, and/or a method of treatment comprising administering said substituted pyrrolidine compound, a method for the manufacture of said substituted pyrrolidine compound, and novel intermediates and partial steps for its synthesis.

The present invention provides especially compounds of the formula I

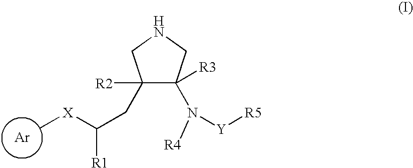

wherein

R¹ is unsubstituted or substituted alkyl or substituted or unsubstituted cycloalkyl;

R² is hydrogen, alkoxy, alkyl, hydroxy or halogen;

R³ is hydrogen or alkyl;

R⁴ is hydrogen, unsubstituted or substituted alkyl or substituted or unsubstituted cycloalkyl;

R⁵ is unsubstituted or substituted alkyl, substituted or unsubstituted heterocyclyl, unsubstituted or substituted or unsubstituted aryl, or substituted or unsubstituted cycloalkyl;

X is CH₂ or O;

Y is —(CO)—, —S(O)₂— or —C(O)O—; and

Ar is unsubstituted or substituted mono- or bicyclic aryl or unsubstituted or substituted mono- or bicyclic aromatic heterocyclyl;

or a salt thereof.

The compounds of the present invention exhibit inhibitory activity on the natural enzyme renin. Thus, compounds of formula I may be employed for the treatment (this term also including prophylaxis) of one or more disorders or diseases selected from, inter alia, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo. If not explicitly or implicitly stated otherwise, halo can also stand for more than one halogen substitutent in moieties such as alkyl, alkanoyl and the like (e.g. in trifluoromethyl, trifluoroacetyl).

Unsubstituted or substituted aryl preferably is a is mono- or polycyclic, especially monocyclic, bicyclic, tricyclic aryl with 6 to 22 carbon atoms, especially phenyl, naphthyl, indenyl or fluorenyl, and is unsubstituted or substituted by one or more, especially one to three, moieties, preferably independently selected from the group consisting of:
a substituted of the formula —(C₀-C₇-alkylene)-(X)ᵣ—C₁-C₇-alkylene-(Y)ₛ—C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-d-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl; C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl;
C₂-C₇-alkenyl, C₂-C₇-alkinyl, phenyl, naphtyl, heterocyclyl, especially as defined below for heterocyclyl, preferably selected from pyrrolyl, furanyl, thienyl, pyrimidine-2,4-dione-1-, -3- or -5-yl and benzo[1,3]-dioxolyl, phenyl- or naphthyl- or heterocyclyl-C₁-C₇-alkyl wherein heterocyclyl is as defined below, preferably selected from pyrrolyl, furanyl, thienyl and benzo[1,3]-dioxolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N—) di-(C₁-C₇-alkyl)-amino-C₁-C₇-alkylcarbonyl, C₁-C₇-alkanoylamino-C₁-C₇-alkylcarbonyl, halo-C₁-C₇-alkoxycarbonyl, phenyl- or naphthyloxycarbonyl, phenyl- or naphthyl-C₁-C₇-alkoxycarbonyl, (N,N—) di-(C₁-C₇-alkyl)-amino-C₁-C₇-alkoxycarbonyl, carbamoyl, N-mono or N,N-di-(naphthyl- or phenyl-)-aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkenylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N—) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl.

Unsubstituted or substituted heterocyclyl is a mono- or bicyclic, unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, S(═O)— or S—(═O)₂—) which is unsubstituted or substituted by one or more, e.g. up to three, substitutents preferably independently selected from the substitutents mentioned above for aryl and from oxo. Preferably, unsubstituted or substituted heterocyclyl is selected from the following moieties:

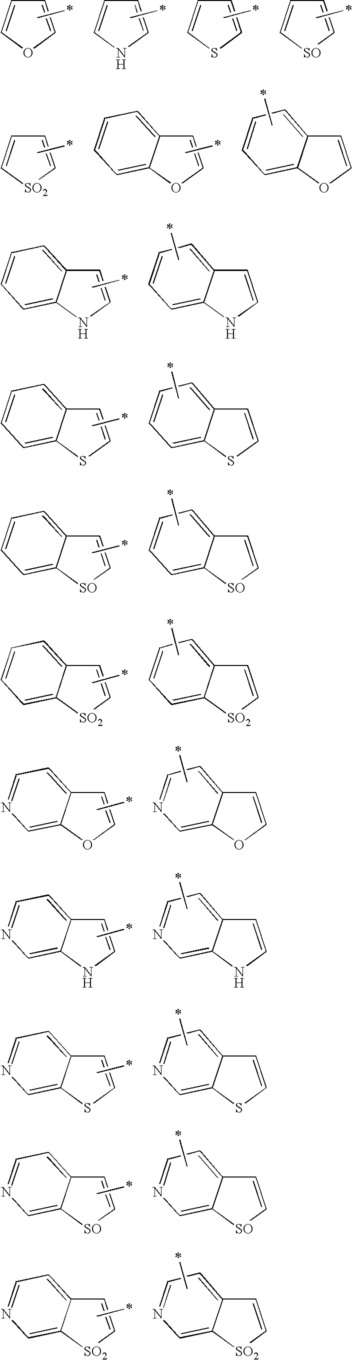

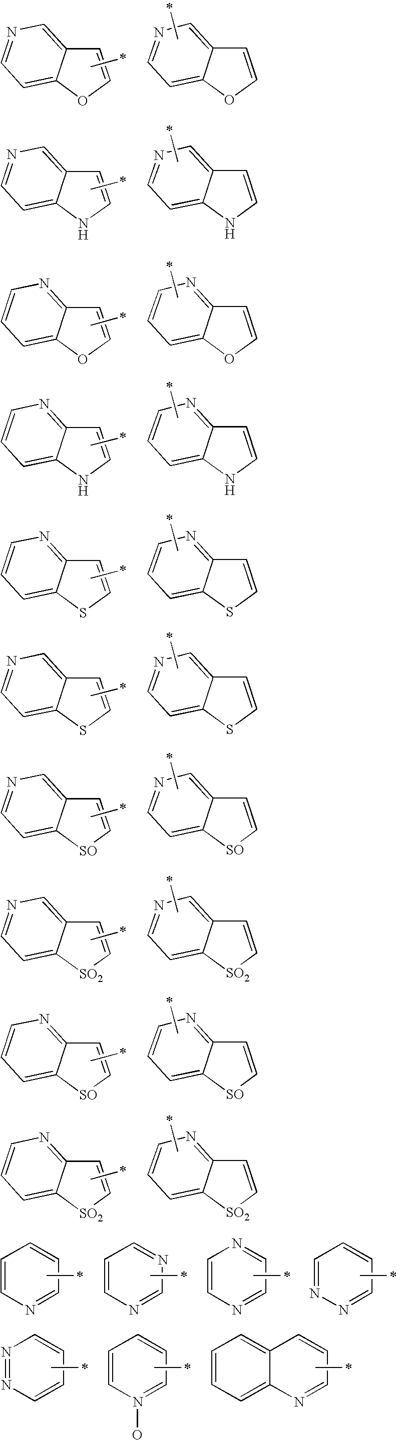

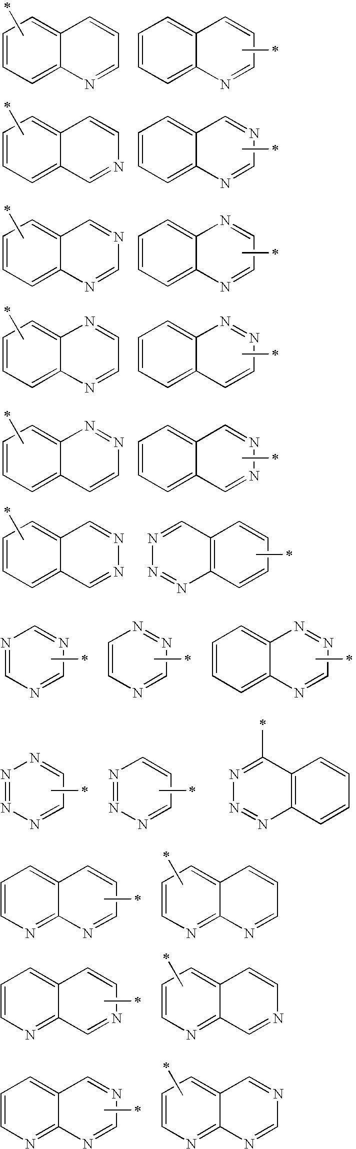

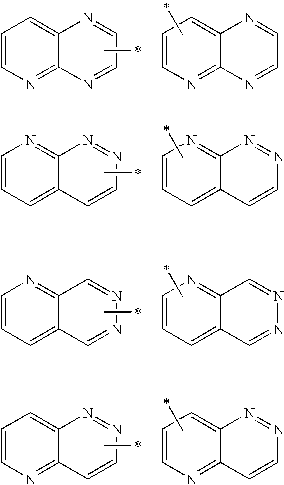

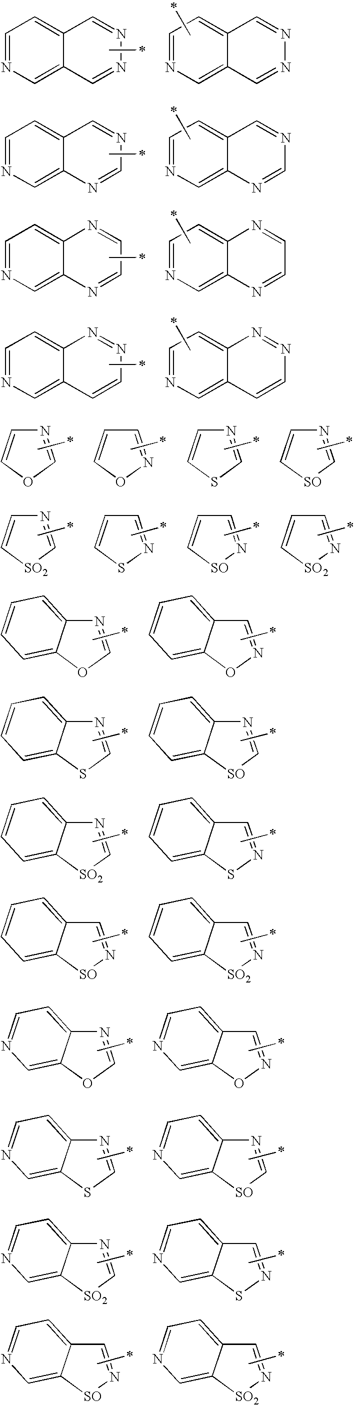

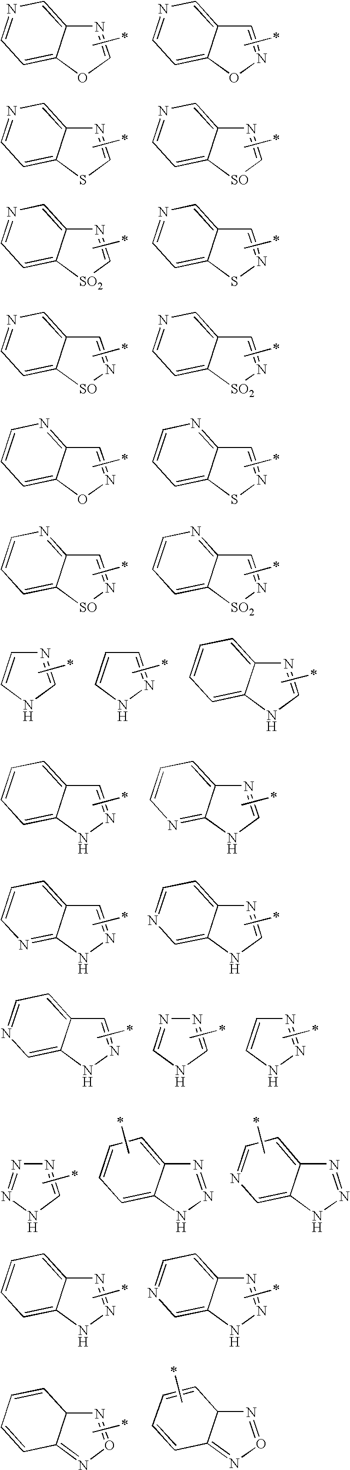

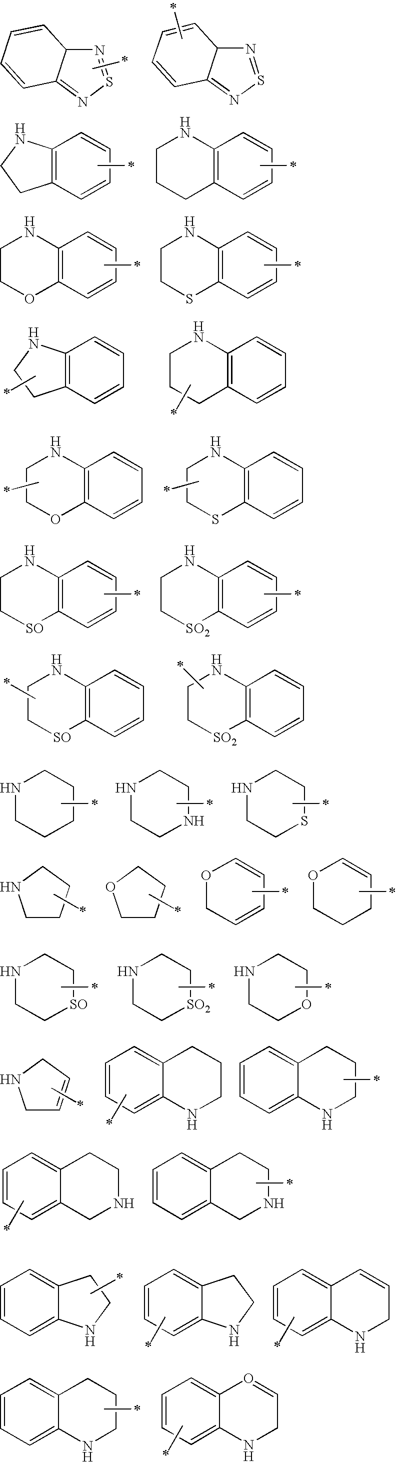

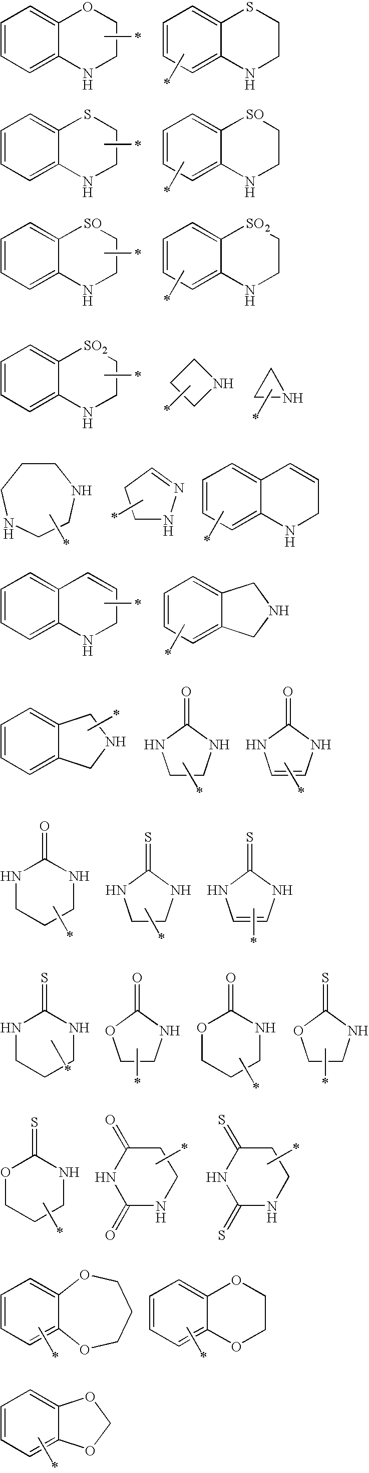

where in each case where an NH is present the bond with the asterisk connecting the respective heterocyclyl moiety to the rest of the molecule the H may be replaced with said bond and/or the H may be replaced by a substituent, and one or more substituents may be present as just described.

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkinyl), and is unsubstituted or substituted by one or more, e.g. one to three substitutents preferably independently selected from those mentioned above as substituents for aryl.

Unsubstituted or substituted alkyl is preferably d-do-alkyl, more preferably C₁-C₇-alkyl, that is straight-chained or branched (one or, where appropriate, more times), which is unsubstituted or substituted by one or more, e.g. up to three moieties selected from unsubstituted or substituted aryl as described above, especially phenyl or naphthyl each of which is unsubstituted or substituted as described above for unsubstituted or substituted aryl, unsubstituted or substituted heterocycyclyl as described above, especially pyrrolyl, furanyl, thienyl, pyrimidine-2,4-dione-1-, -2-, -3- or -5-yl or benzo[1,3]dioxolyl, which heterocyclyl is unsubstituted or substituted as described above for unsubstituted or substituted heterocyclyl; unsubstituted or substituted cycloalkyl as described above, especially cyclopropyl, cyclobutyl, cyclopentyl or cyclohexyl each of which is unsubstituted or substituted as described above for unsubstituted or substituted cycloalkyl; C₂-C₇-alkenyl, C₂-C₇-alkinyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, hydroxy-C₁-C₇-alkylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl, hydroxy-C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxy-carbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkinylene, —C₁-C₇-alkylenedioxy, sulfenyl, (—S—OH) sulfonyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl, N-mono or N,N-di-(C₁-C₇-alkyl, phenyl-, naphthyl, phenyl-C₁-C₇-alkyl or naphthyl-C₁-C₇-alkyl)-aminosulfonyl, N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)-aminocarbonylamino and N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)aminosulfonylamino. In cases where unsubstituted or substituted heterocyclyl-alkyl, unsubstituted or substituted aryl-alkyl or unsubstituted or substituted cycloalkyl-alkyl-moieties are mentioned as substituents, the definition of unsubstituted or substituted alkyl relates to such moieties which, in addition to unsubstituted or substituted heterocyclyl, aryl or cycloalkyl comprise at least one further and different moiety (especially from those mentioned in this paragraph) as alkyl substitutent.

In substituted or unsubstituted alkylsulfonyl, substituted or unsubstituted alkyl is preferably as defined above for unsubstituted or substituted alkyl.

In substituted or unsubstituted arylsulfonyl, substituted or unsubstituted aryl is preferably as defined above for unsubstituted or substituted aryl.

In substituted or unsubstituted heterocyclylsulfonyl, substituted or unsubstituted heterocyclyl is preferably as defined above for unsubstituted or substituted heterocyclyl.

In substituted or unsubstituted cycloalkylsulfonyl, unsubstituted or substituted cycloalkyl is preferably as defined above for unsubstituted or substituted cycloalkyl.

Hereinbefore and hereinafter, lower radicals and compounds are to be understood as being, e.g., those having up to and including 7 carbon atoms, preferably up to and including 4 carbon atoms.

In all definitions above it goes without saying that only stable compounds the person having skill in the art will, without undue experimentation or considerations, be able to recognize are important (e.g. those that are sufficiently stable for the manufacture of pharmaceuticals, e.g. having a half-life of more than 30 seconds) and thus are preferably encompassed by the present claims and that only chemically feasible bonds and substitutions are encompassed, as well as tautomeric forms where present.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-di-methylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” and “intermediates” hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean one (preferred) or more single compound(s), salt(s), pharmaceutical preparation(s), disease(s), disorders) or the like, where the singular or the indefinite article (“a”, “an”) is used, this is intended to include the plural or preferably the singular.

The compounds of the present invention possess two or more asymmetric centers depending on the choice of the substituents. The preferred absolute configuration at the C-3 and C-4 asymmetric centers is maintained throughout the specification and the appended claims as indicated herein-above. However, any possible diastereoisomers, enantiomers and geometric isomers, and mixtures thereof, e.g., racemates, are encompassed by the present invention.

As described herein above, the present invention provides 3,4-disubstituted pyrrolidine derivatives of formula I, these compounds for use in the (prophylactic and/or therapeutic) treatment of a disease (=condition, disorder) in a warm-blooded animal, especially a human, preferably of a disease dependent on (especially inappropriate) renin activity, a pharmaceutical composition comprising a compound of the formula I, methods for preparing said compound or pharmaceutical preparation, and methods of treating conditions dependent on (especially inappropriate) renin activity by administration of a therapeutically effective amount of a compound of the formula I, or a pharmaceutical composition thereof.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration, other circumstances that make the activity of renin relatively too high, such as other mechanisms leading to blood pressure increase, and/or the like) and/or leads to or supports a renin dependent disease or disorder as mentioned above and below, e.g. by renin activity the reduction of which has beneficial effects in the given disease. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore “dependent” has to be read as “dependent inter alia”, (especially in cases where a disease or disorder is really exclusively dependent only on renin) preferably as “dependent mainly”, more preferably as “dependent essentially only”.

Where a disease or disorder dependent on inappropriate activity of a renin is mentioned (such in the definition of “use” in the following paragraph and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural renin and/or one or more altered or mutated forms (including alleles or single nuclear polymorphism forms thereof). Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more disease or disorder mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate.

Highly preferred is a compound of the formula IA with the following configuration:

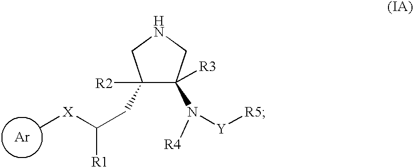

Preferred is a compound of the formula IB with the following configuration:

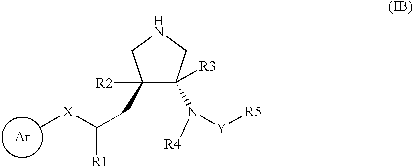

Preferred is also a compound of the formula IC with the following configuration:

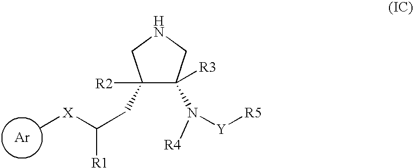

Preferred is also a compound of the formula ID with the following configuration:

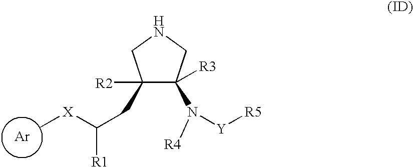

Preferred is also a compound of the formula IE with the following configuration:

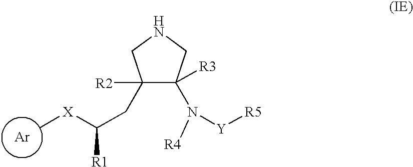

Most preferred is a compound of the formula IF with the following configuration:

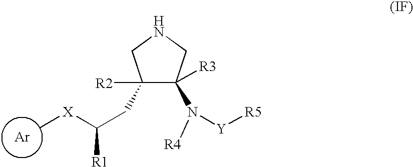

In each of the formulae IA, IB, IC, ID, IE and IF, the moieties R¹, R², R³, R⁴, R⁵, X, Y and Ar are as defined hereinbefore or preferably hereinafter.

The formula IA, IB, IC, ID, IE or IF can replace formula I wherever a compound of the formula I (including a salt thereof) is mentioned hereinbefore or hereinafter; also, the corresponding intermediates are preferred.

The following preferred embodiments of the moieties and symbols in formula I can be employed independently of each other to replace more general definitions and thus to define specially preferred embodiments of the invention, where the remaining definitions can be kept broad as defined in embodiments of the inventions defined above of below.

### Preferred Definitions for R1

R1 is preferably unsubstituted or substituted alkyl or substituted or unsubstituted cycloalkyl, whereby suitable substitutents include O—C₁-C₇-alkyl, halo, hydroxy, unsubstituted or substituted, preferably substituted, phenyl, unsubstituted or substituted, preferably substituted, naphthyl, unsubstituted or substituted, preferably substituted, phenyl- or naphthyloxy, unsubstituted or substituted, preferably substituted, phenyl- or naphthyl-C₁-C₇-alkyloxy, unsubstituted or substituted, preferably substituted, heterocyclycl, unsubstituted or substituted, preferably unsubstituted, cycloalkyl, nitro, amino, amino-C₁-C₇-alkyl, N-mono- or N,N-di-substituted aminocarbonyl, carboxyl, and cyano. More preferably R¹ is unsubstituted.

In one embodiment, R1 is preferably C₁-C₇-alkyl, more preferably C₁-C₄-alkyl, such as methyl, ethyl, n-propyl, isopropyl, n-butyl, isobutyl, sec-butyl and tert-butyl, most preferably isopropyl.

In another preferred embodiment, R1 is preferably C₃-C₁₀-cycloalkyl, more preferably C₃-C₇-cycloalkyl, still more preferably C₃-, C₄-, C₅- or C₆-cycloalkyl, most preferably cyclopropyl.

Most preferably, R1 is isopropyl.

### Preferred Definitions for R2

R2 is preferably hydrogen, hydroxy or halogen, more preferably hydrogen or hydroxyl, most preferably hydrogen.

### Preferred Definitions for R3

R3 is preferably hydrogen.

When one of R2 and R3 are other than hydrogen, such as alkyl, hydroxy or halogen, preferably hydroxyl, then the other is preferably hydrogen.

Preferred Definitions for R4

R4 is preferably hydrogen, unsubstituted or substituted alkyl or substituted or unsubstituted cycloalkyl, whereby suitable substitutents include O—C₁-C₄-alkyl, halo, hydroxy, unsubstituted or substituted, preferably substituted, phenyl, unsubstituted or substituted, preferably substituted, naphthyl, unsubstituted or substituted, preferably substituted, phenyl- or naphthyloxy, unsubstituted or substituted, preferably substituted, phenyl- or naphthyl-C₁-C₇-alkyloxy, unsubstituted or substituted, preferably substituted, heterocyclycl, unsubstituted or substituted, preferably unsubstituted, cycloalkyl, nitro, amino, amino-C₁-C₇-alkyl, N-mono- or N,N-di-substituted aminocarbonyl, carboxyl, and cyano. More preferably R4 is unsubstituted.

In one embodiment, R4 is preferably hydrogen.

In another embodiment, R4 is preferably C₁-C₇-alkyl, more preferably C₁-C₇-alkyl, such as methyl, ethyl, n-propyl, isopropyl, n-butyl, isobutyl, sec-butyl and tert-butyl, most preferably methyl or isopropyl.

In another preferred embodiment, R4 is preferably C₃-C₁₀-cycloalkyl, more preferably C₁-C₇-cycloalkyl, still more preferably C₃-, d-, C₅- or C₆-cycloalkyl, most preferably cyclopropyl.

Most preferably, R4 is hydrogen.

### Preferred Definitions for Y and R5

In one embodiment, Y is preferably —S(O)₂—.

In another embodiment, Y is preferably —C(O)O—.

R5 is preferably unsubstituted or substituted alkyl, substituted or unsubstituted heterocyclyl, unsubstituted or substituted or unsubstituted aryl, or substituted or unsubstituted cycloalkyl, wherein each is unsubstituted or substituted by one or more, e.g. up to three, substitutents selected from the group consisting of

halo, phenyl or naphthyl, heterocyclyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

In a first embodiment R5 is preferably unsubstituted or substituted alkyl.

Preferred examples for alkyl are branched or straight chain C₁-C₇-alkyl which may be substituted or unsubstituted. Preferred examples include methyl, ethyl, isopropyl, n-propyl, n-butyl, sec-butyl or tert-butyl, more preferably methyl, ethyl, isopropyl, or isobutyl most preferably methyl. The alkyl moiety is preferably substituted. When the alkyl moiety is substituted, it is preferably mono-, di- or tri-substituted, more preferably mono-substituted. Suitable substituents for the alkyl moiety are as defined herein, preferably halo, phenyl or naphthyl, heterocyclyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl; and cyano; more preferably halo, phenyl or naphthyl, 5- to 10-membered mono- or bicyclic heterocylyl containing at last one of O, N or S, hydroxy, C₁-C₇-alkoxy, nitro, carboxyl, C₁-C₇-alkoxy-carbonyl, and cyano; most preferably phenyl or tetrahydropyranyl. Whenever phenyl and naphthyl are mentioned as substituents, these may be substituted (mono-, di- or tri-substituted, preferably mono-substituted) or unsubstituted, preferably unsubstituted. Suitable substituents for phenyl, naphthyl or heterocyclyl include C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, d-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl. The hereocyclyl moiety as a substituent of alkyl has preferably the same preferred meaning as set forth below for the second embodiment.

In a second embodiment R5 is preferably unsubstituted or substituted heterocyclyl.

The heterocyclyl moietyl preferably mono- or bicyclic, more preferably bicyclic. Preferred are aromatic ring systems, or partially saturated ring systems, in particular whereby one of the rings is aromatic and the other is saturated or partially saturated, most preferred are saturated. The heterocyclyl moiety has preferably 1, 2 or 3, more preferably 1 or 2, most preferably 2, heteroatoms selected from O, N or S, more preferably O or N. The ring system contains preferably an oxo moiety. Particularly preferred examples include 5- to 10-membered mono- or bicyclic heterocylyl, such as bicyclic 9 or 10-membered rings preferably containing a nitrogen atom, in particular, quinolyl, isoquinolyl, 1,2,3,4-tetrahydro-1,4-benzoxazinyl, 2H-1,4-benzoxazin-3(4H)-only, 3,4-dihydro-1H-quinolin-2-onyl, or 4H-benzo[1,4]thiazin-3-onyl; indolyl, 1H-indazolyl, benzothiophenyl, imidazo[1,2-a]pyridyl or 3H-benzooxazol-2-only; or 5- or 6-membered monocyclic rings containing an O or N atom such as tetrahydrofuranyl, tetrahydropyranyl, furanyl, pyranyl, piperidinyl, pyrrolidinyl, imidazolyl, triazolyl, piperazinyl, morpholinyl, pyrimidinyl or pyridinyl, where each heterocyclyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, —C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl, more preferably C₁-C₇-alkyl, halo, hydroxy-C₁-C₇-alkyl, C₁-C₇-alkoxy-C₁-C₇-alkyl, C₁-C₇-alkanoylamino-C₁-C₇-alkyl, C₁-C₇-alkoxy-C₁-C₇-alkoxy, carbamoyl-C₁-C₇-alkyl. N—C₁-C₇-alkylcarbamoyl-C₁-C₇-alkyl, N—C₁-C₇-haloalkylcarbamoyl-C₁-C₇-alkyl, in particular methyl, pentyl, methoxy-propyl, methoxy-butyl, ethoxy-ethyl, hydroxy-butyl, methoxypropyloxy, F, CH₃—C(O)—NH—CH₂CH₂, NH₂—CO—CH₂CH₂CH₂, N(CH₂CH₃)—CO—CH₂, N(CH₂CH₃)—CO—CH₂. The heterocyclyl moiety is preferably substituted on the N if present. Most preferably, the heterocyclyl is unsubstituted.

In a third embodiment R5 is preferably unsubstituted or substituted aryl.

Preferred examples of aryl include phenyl or naphthyl, more preferably phenyl. When the aryl moiety is substituted, it is preferably mono- or di-substituted. Most preferably aryl is di-substituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, —O—C₁-C₇-alkyl, halo-C₁-C₇-alkyl, halo, cyano, hydroxy-C₁-C₇-alkyl, C₁-C₇-alkoxy-C₁-C₇-alkoxy, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino.

In a fourth embodiment R5 is preferably unsubstituted or substituted cycloalkyl.

Preferred examples of cycloalkyl include d-do-cycloalkyl, more preferably C₁-C₇-cycloalkyl, still more preferably C₃-, d-, C₅- or C₆-cycloalkyl. When the cycloalkyl moiety is substituted, it is preferably mono- or di-substituted. Most preferably cycloalkyl is unsubstituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-CT-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, —O—C₁-C₇-alkyl, halo-C₁-C₇-alkyl, halo, cyano, hydroxy-C₁-C₇-alkyl, C₁-C₇-alkoxy-C₁-C₇-alkoxy, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino.

The first and second embodiment, in particular the first embodiment, are particularly preferred.

In one preferred embodiment, R5 is C₁-C₇-alkyl which is unsubstituted or substituted by one or more, e.g. up to three, substitutents selected from the group consisting of: halo, phenyl or naphthyl, 5- to 10-membered mono- or bicyclic heterocylyl containing at least one heteroatom selected from O, N or S; hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano. Most preferably, R5 is methyl which is unsubstituted or substituted by one or more, e.g. up to three, substitutents selected from the group consisting of phenyl or tetrahydropyranyl.

In another preferred embodiment, R5 is heterocyclyl which is unsubstituted or substituted by one to three substitutents selected from the group consisting of halo, phenyl or naphthyl, heterocyclyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano. Most preferably, R5 is tetrahydropyranyl.

In a preferred embodiment, Y is —(SO₂)— and R5 is unsubstituted or substituted alkyl as defined herein, preferably benzyl.

In a preferred embodiment, Y is —(C═O)O— and R5 is unsubstituted or substituted alkyl as defined herein, preferably benzyl or CH₂-tetrahydropyranyl.

In a preferred embodiment, Y is —(C═O)O— and R5 is unsubstituted or substituted heterocyclyl as defined herein, preferably tetrahydropyranyl.

### Preferred Definitions for X

In a preferred embodiment, X is CH₂

### Preferred Definitions for Ar

Ar is preferably unsubstituted or substituted aryl or unsubstituted or substituted mono- or bicyclic aromatic heterocyclyl, whereby suitable substituents are selected from a substitutent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl; hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl.

More preferably, Ar is phenyl, naphthyl, indolyl, benzimidazolyl, benzofuranyl, quinolinyl, preferably phenyl or indolyl, wherein each is unsubstituted or substituted by one or more, e.g. up to three, substitutents selected from the group consisting of: a substituted of the formula —(C₀-C₇-alkylene)-(X)ᵣ—(C₁-C₇-alkylene)-(Y)ₛ—(C₁-C₇-alkylene)-H where C₀-alkylene means that a bond is present instead of bound alkylene, r and s, each independently of the other, are 0or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl; C₁-C₇-alkoxy, hydroxy-C₁-C₇-alkoxy, C₁-C₇-alkoxy-C₁-C₇-alkoxy, C₁-C₇-alkanoyloxy, mono- or di-(d-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl.

In a first embodiment, Ar is unsubstituted or substituted aryl.

Preferred examples for the aryl moiety are phenyl and naphthy, more preferably phenyl. When the aryl moiety is substituted, it is preferably mono- or di-substituted. Naphthyl is preferably mono-substituted and phenyl is preferably mono- or di-substituted, more preferably di-substituted. Suitable substituents for the aryl moiety are as defined herein:
preferably a substitutent of the formula —(C₁-C₇-alkylene)-(X)ᵣ—(C₁-C₇-alkylene)-(Y)ₛ— (C₀-C₇-alkylene)-H where C₀-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV—wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₁-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl; more preferably, —(C₁-C₇-alkylene)-(X)ᵣ—(C₁-C₇-alkylene)-(Y)ₛ—(C₁-C₇-alkylene)-H, wherein r and s are 0 or 1 and Y and X are independently O, NH or NH—CO—O—, halo-C₁-C₇-alkyl, halo, hydroxy, phenyl- or naphthyloxy, phenyl- or naphthyl-C₁-C₇-alkyloxy, nitro, amino, amino-C₁-C₇-alkyl, carboxyl, and cyano. Preferred examples of —(C₁-C₇-alkylene)-(X)ᵣ—(C₁-C₇-alkylene)-(Y)ₛ—(C₀-C₇-alkylene)-H include —(O or NH)—C₁-C₇-alkyl, —C₁-C₇-alkyl, —(O or NH)—C₁-C₇-alkylene-(O or NH)—C₁-C₇-alkyl, —(O or NH)—C₁-C₇-alkylene-(O or NH)—H, —C₁-C₇-alkylene-(O or NH)—C₁-C₇-alkylene-(O or NH)—C₁-C₇-alkyl, —C₁-C₇-alkylene-(O or NH)—C₁-C₇-alkyl, or —C₁-C₇-alkylene-NH—CO—O—C₁-C₇-alkyl; most preferably —OMe, —OC₃H₆OMe, —NH-butyl, methyl, ethyl, —C₂H₄—NH—CO—OMe, —CH₂OC₂H₄OMe, —OC₂H₄OC₂H₅, —OC₃H₆OH, —C₂H₄OMe, —C₃H₆OMe and —NH—C₃H₆OMe.

Most preferably the aryl moiety is unsubstituted or substituted with OMe and/or —OC₃H₆OMe.

In a second embodiment, Ar is unsubstituted or substituted mono- or bicyclic aromatic hereocyclyl.

The heterocyclyl moiety has preferably 1, 2 or 3, more preferably 1 or 2 heteroatoms selected from O, N or S, more preferably O or N. Particularly preferred examples include pyrrolyl, furanyl, thienyl, pyridyl, pyrimidinyl, indolyl, benzimidazolyl, benzopyrazolyl, benzofuranyl, quinolinyl, more preferably indolyl, benzimidazolyl, benzofuranyl, quinolinyl, most preferably indolyl. When the heterocyclyl moiety is substituted, it is preferably mono-substituted. Suitable substituents for the heterocyclyl moiety are as defined herein, preferably —(C₀-C₇-alkylene)-(X)ᵣ—(C₁-C₇-alkylene)-(Y)ₛ—(C₀-C₇-alkylene)-H, wherein r and s are 0 or 1 and Y and X are independently O, NH or NH—CO—O—, halo-C₁-C₇-alkyl, halo, hydroxy, phenyl- or naphthyloxy, phenyl- or naphthyl-C₁-C₇-alkyloxy, nitro, amino, amino-C₁-C₇-alkyl, carboxyl, and cyano. Preferred examples of —(C₀-C₇-alkylene)-(X)ᵣ—(C₁-C₇-alkylene)-(Y)ₛ—(C₀-C₇-alkylene)-H include —(O or NH)—C₁-C₇-alkyl, —C₁-C₇-alkyl, —(O or NH)—C₁-C₇-alkylene)-(O or NH)—C₁-C₇-alkyl, —(O or NH)—C₁-C₇-alkylene-(O or NH)—H, —C₁-C₇-alkylene-(O or NH)—C₁-C₇-alkylene-(O or NH)—C₁-C₇-alkyl, —C₁-C₇-alkylene-(O or NH)—C₁-C₇-alkyl, or —C₁-C₇-alkylene-NH—CO—O—C₁-C₇-alkyl; more preferably —OMe, —OC₂H₄OMe, —NH-butyl, methyl, ethyl, —C₂H₄—NH—CO—OMe, —CH₂OC₂H₄OMe, —OC₂H₄OC₂H₅, —OC₃H₆OH, —C₂H₄OMe, —C₃H₆OMe and —NH—C₃H₆OMe, yet more preferably —NH-propyl, —C₂H₄OMe and —C₃H₆OMe.

Most preferably the heterocyclyl moiety is unsubstituted or substituted by Me, —C₂H₄OMe or —C₃H₆OMe.

Particularly preferred for Ar is the moiety

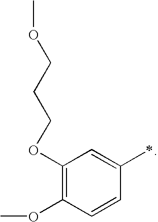

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as their use.

### Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by

A) i) reacting an acid of the formula II,

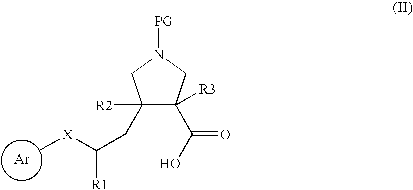

wherein R¹, R², R³, X, and Ar are as defined herein for a compound of the formula I and PG is a protecting group, with diphenylphosphorus azide in the presence of a base and a follower alkylsilyl ethanol to give the corresponding protected amino compound of the formula III.

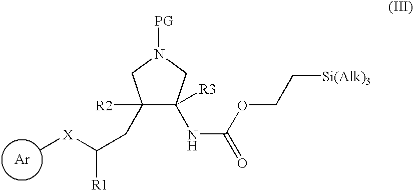

wherein R¹, R², R³, X, Ar and PG are as defined for a compound of the formula II and Alk is C₁₋₄-alkyl;

ii) subsequent removing the tri-lower alkylsilylethoxy group to give an amino compound of the formula IV,

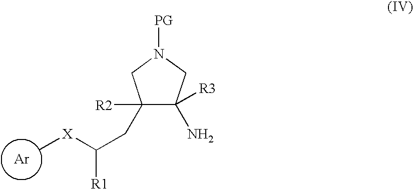

wherein R¹, R², R³, X, Ar and PG are as defined for a compound of the formula II;

iii) reacting a compound of the formula (IV) with a compound of the formula V,

R5—Y-Z (V)

wherein R⁵ and Y have the meanings given above or below for a compound of the formula I and Z is a leaving group to obtain a compound of the formula VI

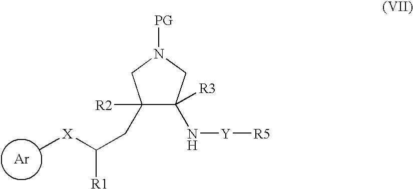

wherein R¹, R², R³, R⁵, X, Y, Ar and PG are as defined for a compound of the formula II or V respectively; and

iv) removing the protecting group PG to obtain a corresponding compound of the formula I, wherein R¹, R², R³, R⁵, X, Y and Ar are as defined herein; or

B) i) oxidizing a compound of the formula VIII,

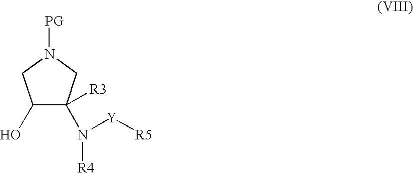

wherein R³, R⁴, R⁵, and Y are as just defined, PG is a protecting group, to obtain a compound of formula IX

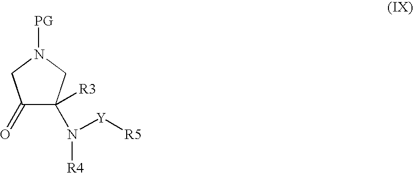

wherein R³, R⁴, R⁵, Y and PG are as just defined;

ii) reacting the compound of formula IX with a metallo reagent of the formula X,

Ar—X—CHR1-CH₂—Mg-Hal (X)

wherein R¹, Ar and X as just defined and Hal is halo, to obtain, upon removal of the protecting group PG, the corresponding compound of the formula I, wherein R² is hydroxyl and R¹, R³, R⁴, R⁵, X, Y and Ar are as defined herein;

and, if desired, subsequent to any one or more of the processes mentioned under (A) to (B) converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers; where in any of the starting materials, in addition to specific protecting groups PG, further protecting groups may be present, and any protecting groups are removed at an appropriate stage in order to obtain the corresponding compound of the formula I, or a salt thereof.

### Preferred Reaction Conditions

The preferred reaction conditions for the reactions mentioned above under A) to B), as well as for the transformations and conversions, are as follows:

In A) the treatment of the compound of the formula II with diphenylphosphorus azide preferably takes place in a suitable solvent, e.g. in dioxane or toluene. In addition a base is present, such as a tertiary nitrogen base, preferably triethylamine. Typically, the reaction proceeds at elevated temperatures, e.g. under reflux. A tri-lower alkylsilyl ethanol, e.g. trimethylsilyl ethanol, is added and the mixture is preferably stirred elevated temperatures, e.g. under reflux, to give the corresponding protected amino compound of the formula III.

The subsequent removal of the tri-lower alkylsilylethoxy group takes place under standard conditions, see also the literature mentioned below under General Process Conditions. For example the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetoneitrile, preferably at elevated temperatures, e.g. under reflux conditions.

The reaction between a compound of the formula IV and a compound of the formula V under A) preferably takes place in the presence of a base, such as a a tertiary nitrogen base, such as triethylamine, in the presence of an appropriate solvent, e.g. a halogenated hydrocarbon, such as methylene chloride, and/or a hydrocarbon, such as toluene, at preferred temperatures between 0° C. and 50° C., e.g. at room temperature. It may also be appropriate to use standard condensation reaction in between an acid, or a reactive derivative thereof, and an amino compound of the formula IV, where among the possible reactive derivatives of an acid reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also be formed in situ. The reaction is carried out by dissolving the compounds of in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine or diisopropylethylamine (DIEA) and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidino-phosphonium-hexafluorophosphate (PyBOP) or 1-(3-dimethylaminopropyl)-3-ethylcarbodiimide hydrochloride/hydroxybenzotriazole (EDCl/HOBT). For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction is preferably carried out under an inert gas, e.g. nitrogen or argon.

The subsequent removal of a protecting group, e.g. PG, such as tert-butoxycarbonyl, benzyl or 2-(trimethylsilyl)-ethoxycarbonyl, takes place under standard conditions, see also the literature mentioned below under General Process Conditions. For example, tert-butoxycarbonyl is removed in the presence of an acid, e.g. a TFA or hydrohalic acid, such as HCl, in an appropriate solvent, e.g. an ether, such as dioxane, at customary temperatures, e.g. at room temperature, the removal of benzyl can be achieved e.g. by reaction with ethylchloroformate or 2-trimethylsilylethyl-chloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., and the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetoneitrile, preferably at elevated temperatures, e.g. under reflux conditions.

The oxidation under B) of a hydroxy compound of the formula VIII to a corresponding oxo compound of the formula IX preferably takes place in the presence of an appropriate oxidant, such as Dess-Martin-periodinane, in an appropriate solvent, e.g. a halogenated hydrocarbon, e.g. methylene chloride, at preferred temperatures from 0° C. to 50° C., e.g. at room temperature. The optional subsequent conversion of an oxo group into a thioxo group (═S) can take place in the presence of Lawesson's reagent or under under customary thionation conditions, the conversion of oxo into an (unsubstituted or substituted) imino by reaction with protected ammonia (for unsubstituted imino) or a primary amine corresponding to a substituted imino to be introduced under customary Schiff base formation conditions.

The coupling under B) between a metallo reagent of the formula X and a compound of the formula IX takes place under customary reaction conditions, e.g. under Grignard coupling conditions, in an appropriate solvent, e.g. an ether, such as diethyl ether, at preferred temperatures in the range from −100 to −50° C., e.g. at −80 to −70° C. Removal of protecting groups takes place preferably as described under A).

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required after removal of protecting groups.

For example, a lower alkoxy (especially methoxy) group present as a substituent of an aryl moiety in a compound of the formula I can be converted into the corresponding hydroxy substituent by reaction, e.g., with boron tribromide in an appropriate solvent, e.g. a halogenated hydrocarbon, at preferred temperatures in the range from −100 to −50° C., e.g. at −80 to −70° C., yielding the corresponding hydroxy compound of the formula I.

A cyano group present as substituent on a compound of the formula I can be converted into an aminomethyl group e.g. by hydrogenation in the presence of a catalyst, such as a transition metal catalyst, e.g. Raney-Nickel, under customary conditions, e.g. in an alcohol, such as methanol, at preferred temperatures between 0° C. and 50° C., e.g. at room temperature, to yield the corresponding amino compound of the formula I, yielding a corresponding compound of the formula I.

An amino group present as a substituent on a compound of the formula I can be converted into an acyl(especially lower-alkanoyl)-amino group e.g. by acylation with a carbonic or sulfonic acid, or a reactive derivative thereof, e.g. the corresponding acid halogenide, such as the acid chloride, or under in situ formation of the corresponding active derivative, under conditions analogous to those described above under A), yielding the corresponding acylamino compound of the formula I.

An amino group present as a substituent on a compound of the formula I can be converted into an N,N-di-(C₁-C₇-alkyl)- or N,N-di-(phenyl- or naphthyl-C₁-C₇-alkyl)-amino group by alkylation e.g. with a corresponding N,N-di-(C₁-C₇-alkyl)- or N,N-di-(phenyl- or naphthyl-C₁-C₇-alkyl)-halogenide, e.g. -chloride or -bromide, or by reductive amination with a corresponding oxo compound (wherein one of the methylene groups in the C₁-C₇-alkyl-comprising compound used as precursor carries oxo instead of two hydrogen atoms) under conditions of reductive amination, yielding a corresponding compound of the formula I. This reaction preferably takes place under customary conditions for reductive amination, e.g. in the presence of an appropriate reducing (e.g. hydrogenation) agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature.

A nitro group present as substituent on a compound of the formula I can be converted into an amino group e.g. by hydrogenation in the presence of a catalyst, such as a transition metal catalyst, e.g. Raney-Nickel, under customary conditions, e.g. in an alcohol, such as methanol, at preferred temperatures between 0° C. and 50° C., e.g. at room temperature, to yield the corresponding amino compound of the formula I, yielding a corresponding compound of the formula I.

A hydroxy group present as a substituent in a compound of the formula I can be converted into an alkylated or acylated hydroxy group, e.g. C₁-C₇-alkoxy-C₁-C₇-alkoxy, C₁-C₇-alkoxy or phenyl- or naphthyl-C₁-C₇-alkyloxy, by reaction with a corresponding alkylhalogenide or acyl-halogenide, e.g. a C₁-C₇-alkoxy-C₁-C₇-alkylchloride or -bromide, a C₁-C₇-alkylchloride or -bromide or a phenyl- or naphthyl-C₁-C₇-alkyl-chloride or -bromide, under appropriate customary substitution reaction conditions, e.g. in the presence of a base, such as an alkali metal carbonate, e.g. potassium carbonate, or a strong base, such as an alkali metal hydride, e.g. sodium hydride, in an appropriate solvent, e.g. an amide, such as dimethylformamide, at preferred temperatures from 0 to 100° C., e.g. from room temperature to 80° C., yielding a corresponding compound of the formula I.

An imino group in a compound of the formula I, e.g. —NH— as part of a substituent in a compound of the formula I comprising an N-heterocyclic moiety, can be transformed into a C₁-C₇-alkoxy-C₁-C₇-alkylimino group by reaction with a C₁-C₇-alkoxy-C₁-C₇-alkylhalogenide, e.g. chloride or bromide, under reaction conditions as described in the directly preceding paragraph, yielding a corresponding compound of the formula I.

An amino group in a compound of the formula I can be converted into an unsubstituted or substituted alkylamino (e.g. C₁-C₇-alkylamino, such as isopropylamino), unsubstituted or substituted cycloalkylamino (e.g. cyclohexylamino), unsubstituted or substituted aryl-alkyl-amino, unsubstituted or substituted heterocyclyl-alkylamino, unsubstituted or substituted cycloalkyl-alkylamino, alkyloxycarbonylamino, alkylcarbonylamino, substituted or unsubstituted alkylsulfonylamino, substituted or unsubstituted arylsulfonylamino (such as C₁-C₇-alkyl-phenylsulfonyl, e.g. tosyl), substituted or unsubstituted heterocyclylsulfonylamino or substituted or unsubstituted cycloalkylsulfonylamino by reaction with the corresponding unsubstituted or substituted alkane, unsubstituted or substituted cycloalkane, unsubstituted or substituted aryl-alkane, unsubstituted or substituted heterocyclyl-alkane, unsubstituted or substituted cycloalkyl-alkane carrying a keto group instead of a methylene or a formyl group instead of a methyl in the alkyl part, under customary reaction conditions for reductive amination, e.g. as described above under B) (i); or by reaction with a substituted or unsubstituted alylsulfonylhalogenide, substituted or unsubstituted arylsulfonylhalogenide, substituted or unsubstituted heterocyclylsulfonylhalogenide or substituted or unsubstituted cycloalkylsulfonylhalogenide under customary reaction conditions, e.g. in the presence of a tertiary amine, such as triethylamine, in an appropriate solvent, e.g. a halogenated hydrocarbon, such as methylene chloride, at preferred temperatures from 0° C. to 50° C., e.g. at room temperature; yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by chromatography, for example by HPLC, using chromatographic substrates with chiral ligands.

Intermediates and final products can be worked up and/or purified according to customary methods, e.g. using chromatographic methods, distribution methods, (re-) crystallization, and the like.

### Starting Materials

Starting Materials, including intermediates, for compounds of the formula I, can be prepared, for example, according to methods that are known in the art, according to methods described in the examples or methods analogous to those described in the examples, and/or they are known or commercially available.

In the subsequent description of starting materials and intermediates and their synthesis, R¹, R², R³, R⁴, R⁵, X, Y, Ar and PG have the meanings given above or in the Examples for the respecive starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”.

A compound of the formula II can, for example, be obtained by reacting a compound of the formula XI,

PG-NH—CH₂—CHR³—CN (XI)

wherein PG is a protecting group, especially benzyl, with a compound of the formula XII,

Ar—X—CHR1-CH═CR²—CH₂-Hal (XII)

wherein Hal is halo, such as bromo, or a different leaving group, such as tosyl, in the presence of a base, such as an alkali metal hydroxide, e.g. NaOH, and e.g. benzyl-tri-(N-butyl)ammonium bromide, in an appropriate solvent, e.g. a halogenated hydrocarbon, such as methylene chloride, and/or water, preferably at a temperature from 10 to 50° C., e.g. 40° C., treating the resulting compound of the formula XIII

Ar—X—CHR1-CH═CR²—CH₂—N(PG)-CH₂—CHR3-CN (XIII)

wherein the substituents have the meanings just described in the presence of a strong base, such as sodium hydride, in an appropriate solvent, e.g. hexamethylphosphoroamide, at preferred temperatures between −10 and 40° C., thus obtaining a compound of the formula XIV,

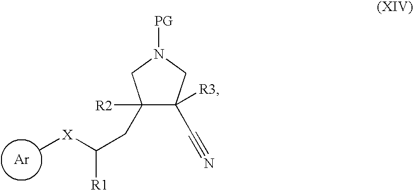

which is then hydrolyzed, e.g. in the presence of a hydrohalic acid, such as HCl, in an appropriate solvent, e.g. acetic acid, water or a mixture thereof, at elevated temperatures, e.g. under reflux, to the corresponding compound of the formula II.

A starting material of the formula II can also be obtained by reacting a compound of the formula XV,

Ar—X—CHR1-CH₂—CHO (XV)

with a compound of the formula XVI,

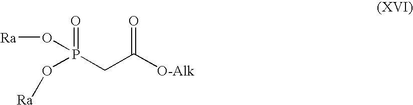

wherein Ra is ethyl or 2,2,2-trifluoroethyl and Alk is lower alkyl, in the presence of a strong base, e.g. sodium hydride e.g. in tetrahydrofurane at preferred temperatures in the range from −10 to 40° C., or in the presence of potassium hexamethyldisiliazane and a crown ether, e.g. 18-crown-6, e.g. in tetrahydrofurane and/or toluene at low temperatures, e.g. from −90 to −70° C., to give a compound of the formula XVII,

Ar—X—CHR1-CH₂—CH═CH—COOAlk (XVII)

which compound is then reacted with a compound of the formula XVIII,

(H₃C)₃Si—CH₂—N(PG)-CH₂O—CH₃ (XVIII)

wherein PG is a protecting group as defined e.g. for a compound of the formula II, in the presence of an acid, e.g. trifluoroacetic acid, in an appropriate solvent, e.g. toluene, at preferred temperatures between −10 and 40° C., to give a compound of the formula XIX,

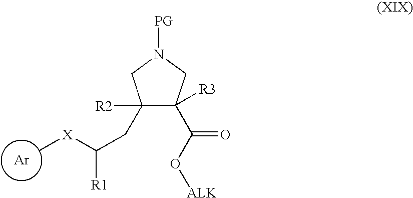

(if desired, the protecting group PG may be replaced by a different protecting group, e.g. benzyl by tert-butoxycarbonyl), and then hydrolysis to remove the Alk-group to give the corresponding free acid of the formula II.

In all formulae above where present, the central pyrrolidine and its substituents at positions 3 and 4 may be present in any one or more of the following configurations, and/or mixtures of the corresponding isomers may be formed and/or separated into the individual isomers at appropriate stages:

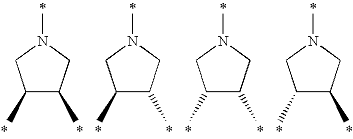

wherein the left lower bond is also on the left side in any of the formulae intermediates or starting materials as shown above or final products of the formula I, the right lower bond on the right side.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition. Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the re-agents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetoneitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### Pharmaceutical Use, Pharmaceutical Preparations and Methods

As described above, the compounds of the present invention are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intraocular pressure, glaucoma, abnormal vascular growth, hyperaldosteronism, cognitive impairment, alzheimers, dementia, anxiety states and cognitive disorders, and the like.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders and the like.

Thus, the pharmacologically active compounds of the invention may be employed in the manufacture of pharmaceutical compositions comprising an effective amount thereof in conjunction or admixture with excipients or carriers suitable for either enteral or parenteral administration. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbents, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable sub-stances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with earner. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052. SB-216763, N,N-57-05441 and N,N-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;
b) hypolipidemic agents such as 3-hydroxy-3-methyl-glutaryl coenzyme A (HMG-CoA) reductase inhibitors, e.g., lovastatin, pravastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) anti-obesity agents such as orlistat; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as dig-oxin, dobutamine and milrinone; calcium channel blockers such as amLodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention alone or in combination with a therapeutically effective amount of another therapeutic agent, preferably selected from anti-diabetics, hypolipidemic agents, anti-obesity agents or anti-hypertensive agents, most preferably from antidiabetics, anti-hypertensive agents or hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angio-plasty, raised intra-ocular pressure, glaucoma, abnormal vascular growth, hyperaldosteronism, cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material therefore.

The present invention further provides a method for the prevention and/or treatment of conditions mediated by (especially inappropriate) renin activity, which comprises administering a therapeutically effective amount of a compound of the present invention to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity.

Preferably, the condition associated with (especially inappropriate) renin activity is selected from hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Finally, the present invention provides a method or use which comprises administering a compound of formula I in combination with a therapeutically effective amount of an anti-diabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting proper-ties. In particular, they inhibit the action of the natural enzyme renin. Renin passes from the kidneys into the blood where it effects the cleavage of angiotensinogen, releasing the deca-peptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably show IC₅₀ values in the range from 10 nM to 20 nM

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably show IC₅₀ values in the range from 10 nM to 20 μM.

In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-×5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably show IC₅₀ values in the range from 10 nM to 20 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably show IC₅₀ values in the range from 10 nM to 20 μM.

In animals deficient in salt, renin inhibitors bring about a reduction in blood pressure. Human renin may differ from the renin of other species. In order to test inhibitors of human renin, primates, e.g. marmosets (Callithrix jacchus) may be used, because human renin and primate renin are substantially homologous in the enzymatically active region. Inter alia the following in vivo tests may be used:

Compounds can be tested in vivo in primates as described in the literature (see for example by Schnell C R et al. Measurement of blood pressure and heart rate by telemetry in conscious, unrestrained marmosets. Am J Physiol 264 (Heart Circ Physiol 33). 1993:1509-1516; or Schnell C R et al. Measurement of blood pressure, heart rate, body temperature, ECG and activity by telemetry in conscious, unrestrained marmosets. Proceedings of the fifth FELASA symposium: Welfare and Science. Eds BRIGHTON. 1993.

The following Examples, while representing preferred embodiments of the invention, serve to illustrate the invention without limiting its scope.

### ABBREVIATIONS

Ac acetyl
AcOH acetic acid
DIBAL-H diisobutylaluminium hydride
4-DMAP 4-dimethylamino-pyridine
DMF dimethylformamide
DMPU 1,3-dimethyl-3,4,5,6-tetrahydro-2(1H)-pyrimidinone
DMSO dimethylsulfoxide
DPPA diphenylphosphoryl azide
EDCl 1-(3-dimethylaminopropyl)-3-ethylcarbodiimide hydrochloride
EtOAc ethyl acetate
Et₃N triethylamine
EtOH ethanol
Flow flow rate
h hour(s)
HMPA hexamethylphosphoroamide
HOBt 1-hydroxybenzotriazole
HPLC High Performance Liquid Chromatography
iPrOH isopropanol
L liter(s)
KHMDS potassium hexamethyldisilazane
LC-MS Liquid Chromatography/Mass Spectrometry
LDA lithium diisopropylamine
Me methyl
MeI methyl iodide
MeOH methanol
MesCl methanesulfonyl chloride
Min minute(s)
mL milliliter
MS Mass Spectrometry
NMM 4-methylmorpholine
NMR Nuclear Magnetic Resonance
Pd/C palladium on charcoal
RT room temperature
TEAF tetraethylammonium fluoride
t-BuOK potassium tert-butoxide
TEA triethylamine
TFA trifluoroacetic acid
TMSE 2-(trimethylsilyl)ethanol
THF tetrahydrofurane
RP reverse phase
TLC Thin Layer Chromatography
tᵣ retention time

### TRADEMARKS

Celite=Celite® (the Celite Corporation)=filtering aid based on diatomaceous earth
Nucleosil=Nucleosil®, trademark of Machery & Nagel, Duren, FRG for HPLC materials

Temperatures are measured in degrees Celsius. Unless otherwise indicated, the reactions take place at RT.

TLC conditions: R_{f} values for TLC are measured on 5×10 cm TLC plates, silica gel F₂₅₄. Merck, Darmstadt, Germany.

The general procedure to produce compounds of formula I is exemplified in Schemes 1 and 2 below and as described in more detail in the examples.

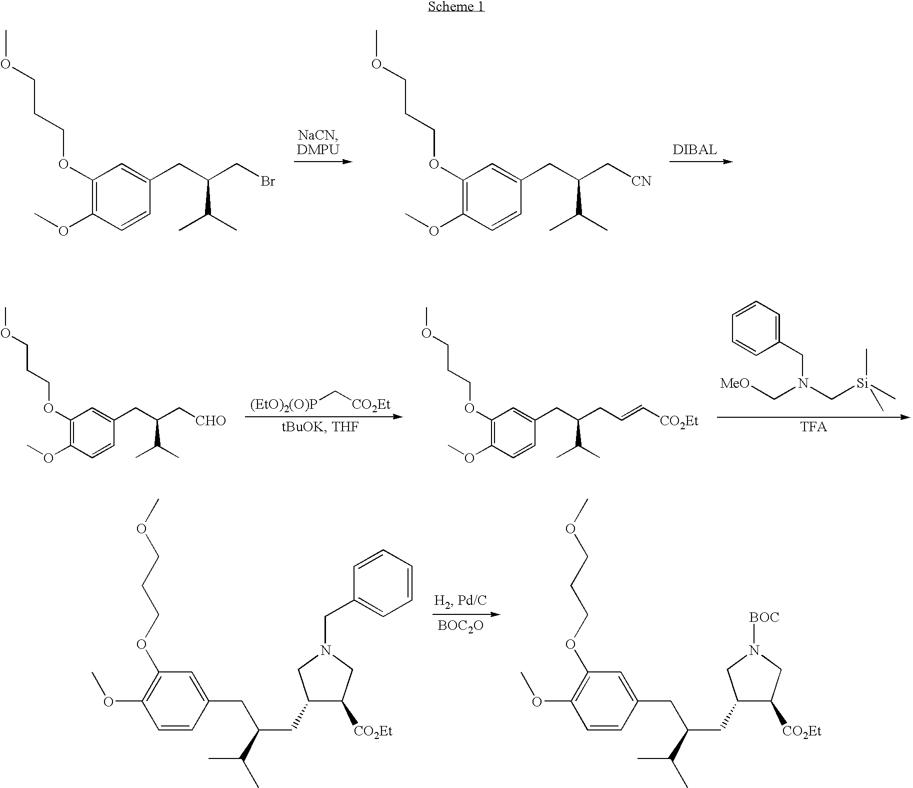

### Example 1

### (3S*,4R*)-4-{(R)-2-[4-Methoxy-3-(3-methoxy-propoxy)-benzyl]-3-methyl-butyl}-pyrrolidin-3-yl)-carbamic acid benzyl ester

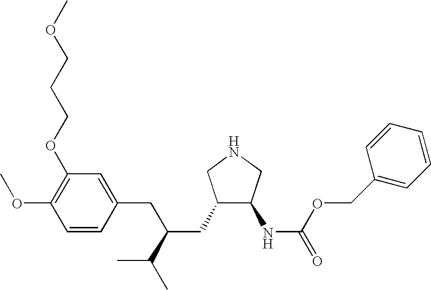

To (3S*,4R*)-3-Benzyloxycarbonylamino-4-{(R)-2-[4-methoxy-3-(3-methoxy-propoxy)-benzyl]-3-methyl-butyl}-pyrrolidine-1-carboxylic acid tert-butyl ester (250 mg, 0.393 mmol) is added a 4M HCl solution in dioxane (0.98 mL, 3.90 mmol), and stirring is continued at room temperature overnight. The mixture is then freeze-dried in high vacuo overnight to give the title compound as its mono hydrochloride salt. TLC, R_{f} (CH₂Cl₂/MeOH/conc. NH₃ 90:10:1)=0.70. RP-HPLC: t_{R}=5.28 min (Nucleosil C₁₈-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 μm). MS: 537.4 [M+H]⁺.

The starting materials are prepared according to Scheme 1 and Scheme 2 as follows:

### A. (S)-3-[4-Methoxy-3-(3-methoxy-propoxy)-benzyl]-4-methyl-pentanenitrile

To a solution of 4-((R)-2-bromomethyl-3-methyl-butyl)-1-methoxy-2-(3-methoxy-propoxy)-benzene, prepared as described in Helv. Chimica Acta 2003, 86, 2848-2870, (30.5 g, 84.9 mmol) in DMPU (450 mL) is added in portions NaCN (17.5 g, 357 mmol) with stirring. The reaction mixture is warmed to 50° C. for 2 h, followed by addition of water after cooling to ambient temperature. The aqueous layer is extracted with EtOAc, and the combined organics are repeatedly washed with water, dried (Na₂SO₄) and concentrated. The crude product is purified by flash chromatography on silica gel (eluent gradient: hexane/EtOAc 85:15 to 70:30) to give the title compound as colorless oil. TLC, R_{f} (hexane/EtOAc 3:1)=0.32. MS: 306.2 [M+H]⁺ and 323.2 [M+18]⁺.

### B. (S)-3-[4-Methoxy-3-(3-methoxy-propoxy)-benzyl]-4-methyl-pentanal

To a solution of (S)-3-[4-methoxy-3-(3-methoxy-propoxy)-benzyl]-4-methyl-pentanenitrile (12.2 g, 39.9 mmol) in toluene (20 mL), cooled to −60° C., is added dropwise with stirring a 1.7M solution of DIBAL-H (32.9 mL, 55.9 mmol). After 15 min at −60° C., the mixture is slowly warmed to ambient temperature with stirring overnight. The mixture is cooled to 0° C., followed by dropwise addition of EtOAc (23 mL). Stirring is continued for 1 h at room temperature, the mixture is again cooled to 0° C., followed by dropwise addition of a saturated aqueous solution of NH₄Cl (108 mL) and, after one additional hour, by addition of 2M H₂SO₄ (108 mL) and diethyl ether (100 mL). After warming to room temperature over 1 h, the layers are separated and the aqueous phase is extracted with diethyl ether. The combined organics are washed with saturated NaHCO₃ and water, dried (Na₂SO₄) and concentrated. The crude product is purified by flash chromatography on silica gel (hexane/EtOAc 3:1) to give the title compound. TLC, R_{f}, (hexane/EtOAc 3:1)=0.35. MS: 326.2 [M+18]⁺.

### C. (R)-5-[4-Methoxy-3-(3-methoxy-propoxy)-benzyl]-6-methyl-hept-2-enoic acid ethyl ester

A solution of triethyl 2-phosphonoacetate (7.41 g, 33.1 mmol) in THF (20 mL) is added dropwise over 5 min to a solution potassium tert-butoxide (3.09 g, 27.5 mmol) in THF (40 mL) under an argon atmosphere. After stirring for 30 min at room temperature, a solution of (S)-3-[4-methoxy-3-(3-methoxy-propoxy)-benzyl]-4-methyl-pentanal (7.08 g, 13.8 mmol) in THF (20 mL) is added dropwise and stirring is continued for 30 min. The mixture is then poured into a diluted aqueous NH₄Cl solution and the water phase is extracted with diethyl ether. The combined organics are washed with saturated aqueous NH₄Cl, dried (Na₂SO₄) and concentrated. The crude material is purified by flash chromatography on silica gel (hexane/EtOAc 8:2) to give the title compound as colorless oil. TLC, R_{f} (hexane/EtOAc)=0.36. MS: 396.2 [M+18]⁺.

### D. (3S*,4S*)-1-Benzyl-4-{(R)-2-[4-methoxy-3-(3-methoxy-propoxy)-benzyl]-3-methyl-butyl}-pyrrolidine-3-carboxylic acid ethyl ester

To a solution of (R)-5-[4-methoxy-3-(3-methoxy-propoxy)-benzyl]-6-methyl-hept-2-enoic acid ethyl ester (5.13 g, 12.6 mmol) in toluene (50 mL), cooled to 0° C., is subsequently added under an argon atmosphere N-methoxy-N-(trimethylsilylmethyl)benzylamine (3.82 g, 15.1 mmol; Lancaster 19412) and a solution of trifluoroacetic acid (0.095 mL, 1.26 mmol) in CH₂Cl₂ (0.5 mL) in a dropwise fashion. Stirring is continued at 0° C. for 30 min and at room temperature overnight. To the reaction mixture is then added a saturated aqueous NaHCO₃ solution, and the water layer is extracted with EtOAc, the combined organics are dried over Na₂SO₄, filtered and concentrated. The residue is purified by flash chromatography (eluent gradient: hexane/EtOAc 3:1 to 2:1) to give the title compound as a mixture of trans-configured diastereomers. Colorless oil. TLC, R_{f} (hexane/EtOAc 3:1)=0.24. MS: 512.2[M+H]⁺.

### E. (3S*,4S*)-4-{(R)-2-[4-Methoxy-3-(3-methoxy-propoxy)-benzyl]-3-methyl-butyl}-pyrrolidine-1,3-dicarboxylic acid 1-tert-butyl ester 3-ethyl ester

A solution of (3S*,4S*)-1-benzyl-4-{(R)-2-[4-methoxy-3-(3-methoxy-propoxy)-benzyl]-3-methyl-butyl}-pyrrolidine-3-carboxylic acid ethyl ester (5.05 g, 9.87 mmol) and di-tert-butyl dicarbonate (2.59 g, 11.8 mmol) in analytical grade EtOH (100 mL) is hydrogenated for 18 h in the presence of catalytic 10% Pd/C (0.5 g; Engelhard 4505) at 25° C. under atmospheric pressure. The reaction mixture is filtered through Celite®, and the combined filtrated are concentrated. Purification by flash chromatography (hexane/EtOAc 3:1) gives the title compound as ca. 1:1 mixture of trans-configured diastereomers. Colorless oil. TLC, R_{f} (hexane/EtOAc 3:1)=0.31. MS: 522.1 [M+H]⁺; 539.1 [M+18]*.

### F. (3S*,4S*)-4-{(R)-2-[4-Methoxy-3-(3-methoxy-propoxy)-benzyl]-3-methyl-butyl}-pyrrolidine-1,3-dicarboxylic acid 1-tert-butyl ester

A solution of (3S*,4S*)-4-{(R)-2-[4-methoxy-3-(3-methoxy-propoxy)-benzyl]-3-methyl-butyl}-pyrrolidine-1,3-dicarboxylic acid 1-tert-butyl ester 3-ethyl ester (2.56 g, 4.91 mmol) in EtOH 50 mL) and 2M NaOH (12.3 mL) is stirred at 50° C. overnight. Volatiles are removed in vacuo and the residue is diluted with water. The aqueous phase is acidified to pH 1 by addition of concentrated HCl and then extracted with CH₂Cl₂. The combined organics are dried (Na₂SO₄), filtered and concentrated to give the title compound as foam. TLC, R_{f} (CH₂Cl₂/MeOH/AcOH 90:10:1)=0.50. RP-HPLC: t_{R}=5.81 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 492.2 [M−H]⁺.

### H. (3S*,4R*)-4-{(R)-2-[4-Methoxy-3-(3-methoxy-propoxy)-benzyl]-3-methyl-butyl}-3-(2-trimethylsilanyl-ethoxycarbonylamino)-pyrrolidine-1-carboxylic acid tert-butyl ester

To a solution of (3S*,4S*)-4-{(R)-2-[4-Methoxy-3-(3-methoxy-propoxy)-benzyl]-3-methyl-butyl}-pyrrolidine-1,3-dicarboxylic acid 1-tert-butyl ester (2.20 g, 4.47 mmol) in toluene (70 mL) are added diphenyl phosphoryl azide (1.54 mL, 7.13 mmol; Fluka 72935) and NEt₃ (1.62 mL, 11.6 mmol) and the reaction mixture is heated for 4 h to reflux. Subsequently, 2-(trimethylsilyl)ethanol (1.27 mL, 8.91 mmol) is added and the mixture is stirred at reflux temperature overnight. After cooling, volatiles are removed in vacuo and the residue is taken up in CH₂Cl₂. The organic layer is washed with water and brine, dried (Na₂SO₄), filtered and concentrated. Purification by flash chromatography on silica gel (hexane/EtOAc 3:1) gives the title compound as colorless oil. TLC, R_{f} (hexane/EtOAc 3:1)=0.22. MS (negative ionization mode): 607.3 [M−H]⁺.

### I. (3S*,4R*)-3-Amino-4-{(R)-2-[4-methoxy-3-(3-methoxy-propoxy)-benzyl]-3-methyl-butyl}-pyrrolidine-1-carboxylic acid tert-butyl ester

A mixture of (3S*,4R*)-{(R)-2-[4-methoxy-3-(3-methoxy-propoxy)-benzyl]-3-methyl-butyl}-3-(2-trimethylsilanyl-ethoxycarbonylamino)-pyrrolidine-1-carboxylic acid tert-butyl ester (1.30 g, 2.14 mmol) and tetraethylammonium fluoride dihydrate (0.79 g, 4.27 mmol) in acetonitrile (20 mL) is heated with stirring at 60° C. overnight. Volatiles are removed in vacuo and the residue is taken up in CH₂Cl₂, followed by washing the organic layer with saturated aqueous NaHCO₃ and saturated NH₄Cl. The organics are dried over Na₂SO₄, filtered and concentrated. After drying in high vacuo, the title compound is obtained as a ca. 1:1 mixture of trans-configured diastereomers. Colorless oil. TLC, R_{f} (CH₂Cl₂/MeOH 97:3)=0.23. RP-HPLC: t_{R}=4.98 and 5.06 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 465.2 [M+H]⁺.

### J. (3S*,4R*)-3-Benzyloxycarbonylamino-4{(R)-2-[4-methoxy-3-(3-methoxy-propoxy)-benzyl]-3-methyl-butyl}pyrrolidine-1-carboxylic acid tert-butyl ester

A mixture of (3S*,4R*)-3-amino-4-{(R)-2-[4-methoxy-3-(3-methoxy-propoxy)-benzyl]-3-methyl-butyl}-pyrrolidine-1-carboxylic acid tert-butyl ester (150 mg, 0.323 mmol), benzyl chloroformate (55 μL, 0.39 mmol) and triethylamine (54 μL, 0.39 mmol) in CH₂Cl₂ (4 mL) is stirred at room temperature overnight. After diluting with CH₂Cl₂, the organic layer is washed with 1M aqueous HCl (5 mL), saturated NaHCO₃ and brine, dried (Na₂SO₄) and concentrated. The residue is purified by flash chromatography on silica gel (eluent gradient:hexane/EtOAc 8:2 to 7:3) to give the title compound as colorless oil. TLC, R_{f} (hexane/EtOAc 1:1)=0.65. RP-HPLC: t_{R}=6.49 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 599.1[M+H]⁺.

### Example 2

### N-((3S*,4R*)-4-{(R)-2-[4-Methoxy-3-(3-methoxy-propoxy)-benzyl]-3-methyl-butyl}-pyrrolidin-3-yl)-C-phenyl-methanesulfonamide

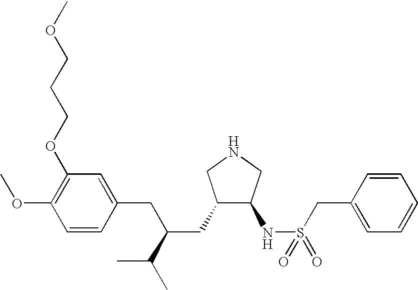

The title compound is prepared by the procedure described in Example 1, starting from (3S*,4R*)-4-{(R)-2-[4-methoxy-3-(3-methoxy-propoxy)-benzyl]-3-methyl-butyl}-3-phenyl-methanesulfonylamino-pyrrolidine-1-carboxylic acid tert-butyl ester (182 mg, 0.294 mmol) and 4M HCl in dioxane (0.74 mL, 2.94 mmol). After freeze-drying in high vacuo overnight the title compound is obtained as its mono hydrochloride salt. White powder. TLC, R_{f} (CH₂Cl₂/MeOH/conc. NH₃ 90:10:1)=0.48. RP-HPLC: t_{R}=4.94 min (Nucleosil C18-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 nm). MS: 519.2 [M+H]⁺.

The starting materials are prepared as follows (Scheme 2):

### A. (3S*,4R*)-4-{(R)-2-[4-Methoxy-3-(3-methoxy-propoxy)-benzyl]-3-methyl-butyl}-3-phenylmethanesulfonylamino-pyrrolidine-1-carboxylic acid tert-butyl ester

A mixture of (3S*,4R*)-3-amino-4-{(R)-2-[4-methoxy-3-(3-methoxy-propoxy)-benzyl]-3-methyl-butyl}-pyrrolidine-1-carboxylic acid tert-butyl ester (175 mg, 0.377 mmol), α-toluenesulfonyl chloride (86 mg, 0.452 mmol) and triethylamine (63 μL, 0.452 mmol) in CH₂Cl₂ (4 mL) is stirred at room temperature overnight. After diluting with CH₂Cl₂, the organic layer is washed with 1M aqueous HCl (5 mL), saturated NaHCO₃ and brine, dried (Na₂SO₄) and concentrated. The residue is purified by flash chromatography on silica gel (eluent gradient: hexane/EtOAc 8:2 to 7:3) to give the title compound as colorless oil. TLC, R_{f} (hexane/EtOAc 1:1)=0.51. RP-HPLC: t_{R}=6.49 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 619.0 [M+H]⁺.

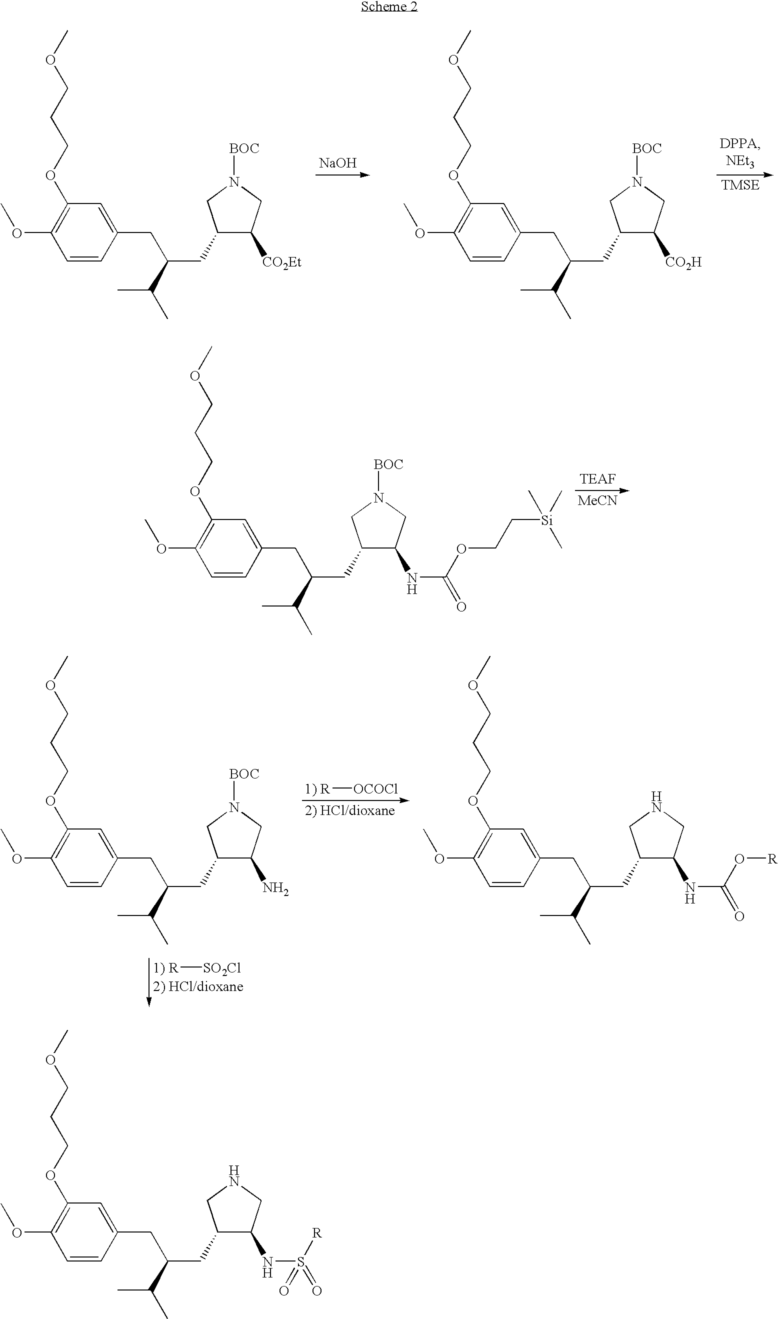

### Example 3

### ((3S*,4R*)-4-{(R)-2-[4-Methoxy-3-(3-methoxy-propoxy)-benzyl]-3-methyl-butyl}-pyrrolidin-3-yl)-carbamic acid tetrahydropyran-4-ylmethyl ester

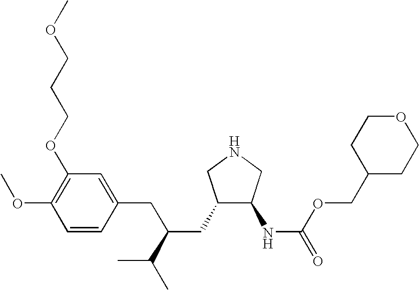

The title compound was prepared in a similar fashion as described in Example 1, starting from (3S*,4R*)-3-amino-4-{(R)-2-[4-methoxy-3-(3-methoxy-propoxy)-benzyl]-3-methyl-butyl}-pyrrolidine-1-carboxylic acid tert-butyl ester. MS: 507.2 [M+H]⁺.

### Example 4

### ((3S*,4R*)-4-{(R)-2-[4-Methoxy-3-(3-methoxy-propoxy)-benzyl]-3-methyl-butyl}-pyrrolidin-3-yl)-carbamic acid tetrahydro-pyran-4-yl ester

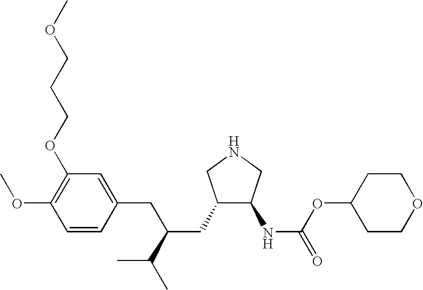

The title compound was prepared in a similar fashion as described in Example 1, starting from (3S*,4R*)-3-amino-4-{(R)-2-[4-methoxy-3-(3-methoxy-propoxy)-benzyl]-3-methyl-butyl}-pyrrolidine-1-carboxylic acid tert-butyl ester. MS: 493.3 [M+H]⁺.

### Example of Formulation 1: Soft Capsules

5000 soft gelatin capsules, each comprising as active ingredient 0.05 g of any one of the compounds of formula I mentioned in any one of the preceding Examples, are prepared as follows:

1. Composition

| | | |
|---|---|---|
| Active ingredient | 250 | g |
| Lauroglycol | 2 | liters |

Preparation process: The pulverized active ingredient is suspended in Lauroglykol® (propylene glycol laurate, Gattefosse S. A., Saint Priest, France) and ground in a wet pulverizer to produce a particle size of about 1 to 3 μm. 0.419 g portions of the mixture are then introduced into soft gelatin capsules using a capsule-filling machine.

### Example of Formulation 2: Tablets Comprising Compounds of the Formula I

Tablets, comprising, as active ingredient, 100 mg of any one of the compounds of formula I in any one of the preceding Examples are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinyl-polypyrrolidone, cross-linked (BASF, Germany). Aerosil® is silicon dioxide (Degussa, Germany).

1. A compound of the formula I 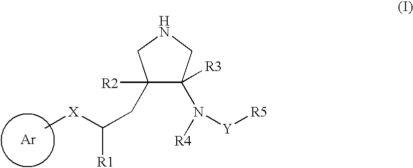
wherein
R¹ is unsubstituted or substituted alkyl or substituted or unsubstituted cycloalkyl;
R² is hydrogen, alkoxy, alkyl, hydroxy or halogen;
R³ is hydrogen or alkyl;
R⁴ is hydrogen, unsubstituted or substituted alkyl or substituted or unsubstituted cycloalkyl;
R⁵ is unsubstituted or substituted alkyl, substituted or unsubstituted heterocyclyl, unsubstituted or substituted or unsubstituted aryl, or substituted or unsubstituted cycloalkyl;
X is CH₂ or O;
Y is —(CO)—, —S(O)₂— or —C(O)O—; and
Ar is unsubstituted or substituted mono- or bicyclic aryl or unsubstituted or substituted mono- or bicyclic aromatic heterocyclyl;
or a salt thereof.

2. A compound of the formula I according to claim 1,
wherein
R¹ is unsubstituted or substituted alkyl or substituted or unsubstituted cycloalkyl;
R² is hydrogen, alkoxy, alkyl, hydroxy or halogen;
R³ is hydrogen or alkyl;
R⁴ is hydrogen, unsubstituted or substituted alkyl or substituted or unsubstituted cycloalkyl;
R³ is unsubstituted or substituted alkyl, substituted or unsubstituted heterocyclyl, unsubstituted or substituted or unsubstituted aryl, or substituted or unsubstituted cycloalkyl;
X is CH₂ or O;
Y is —(CO)—, —S(O)₂— or —C(O)O—; and
Ar is unsubstituted or substituted mono- or bicyclic aryl or unsubstituted or substituted mono- or bicyclic aromatic heterocyclyl;

3. A compound of the formula I according to claim 1, wherein
R¹ is C₁-C₇-alkyl or C₃-C₁₀-cycloalkyl.

4. A compound of the formula I according to claim 1, wherein
R² and R³ are independently of each other hydrogen.

5. A compound of the formula I according to claim 1, wherein
R⁴ is hydrogen or C₃-C₁₀-cycloalkyl.

6. A compound of the formula I according to claim 1, wherein
R⁵ is unsubstituted or substituted alkyl, substituted or unsubstituted heterocyclyl, unsubstituted or substituted or unsubstituted aryl, or substituted or unsubstituted cycloalkyl, wherein each is unsubstituted or substituted or one or more, e.g. up to three, substitutents selected from the group consisting of
halo, phenyl or naphthyl, heterocyclyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

7. A compound of the formula I according to claim 1, wherein
R⁵ is C₁-C₇-alkyl which is unsubstituted or substituted by one or more, e.g. up to three, substitutents selected from the group consisting of
halo, phenyl or naphthyl, 5- to 10-membered mono- or bicyclic heterocylyl containing at least one heteroatom selected from O, N or S; hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

8. A compound of the formula I according to claim 1, wherein
R⁵ is methyl which is unsubstituted or substituted by one or more, e.g. up to three, substitutents selected from the group consisting of
phenyl or tetrahydropyranyl.

9. A compound of the formula I according to claim 1, wherein
R⁵ is heterocyclyl which is unsubstituted or substituted by one to three substitutents selected from the group consisting of halo, phenyl or naphthyl, heterocyclyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl. C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

10. A compound or the formula I according to claim 1, wherein R5 is tetrahydropyranyl which is unsubstituted or substituted by one to three substitutents selected from the group consisting of halo, phenyl or naphthyl, heterocyclyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl, N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

11. A compound of the formula I according to claim 1, wherein
X is CH₂.

12. A compound of the formula I according to claim 1, wherein
Y is —S(O)₂—.

13. A compound of the formula I according to claim 1, wherein
Y is —C(O)O—.

14. A compound of the formula I according to claim 1, wherein
Ar is phenyl, naphthyl, indolyl, benzimidazolyl, benzofuranyl, quinolinyl, each of which is unsubstituted or substituted by one to three substitutents selected from the group consisting of
a substitutent of the formula —(C₀-C₇-alkylene)-(X)ᵣ—(C₁-C₇-alkylene)-(Y)ₛ—(C₀-C₇-alkylene)-H wherein Co-alkylene means that a bond is present instead of bound alkylene, r and s, each independently of the other, are 0 or 1 and each of X and Y, if present and independently of the each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from: C₁-C₇-alkyl, or is phenyl, naphthyl, phenyl- or naphthyl-C₁-C₇-alkyl, and halo-C₁-C₇-alkyl.

15. A compound of the formula I according to claim 1, having the
formula IA, 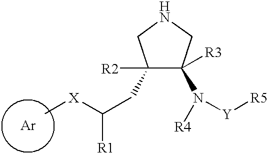
(IA)
or a pharmaceutically acceptable salt thereof.

16. A compound of the formula I according to claim 1, having the formula IB, 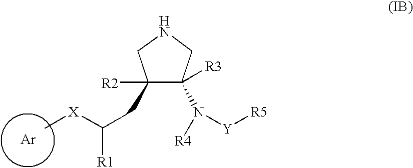
the formula IC, 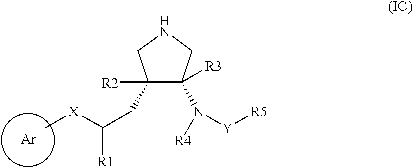
or the formula ID, 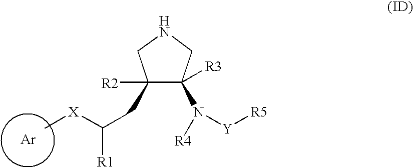
or a pharmaceutically acceptable salt thereof.

17-20. (canceled)

21. A pharmaceutical formulation, comprising a compound of the formula I, or a pharmaceutically acceptable salt thereof, according to claim 1 and at least one pharmaceutically acceptable carrier material.

22. A method of treatment a disease that depends on activity of renin, comprising administering to a warm-blooded animal, especially a human, in need of such treatment a pharmaceutically effective amount of a compound of the formula I, or a pharmaceutically acceptable salt thereof, according to claim 1.

23. A process for the manufacture of a compound of the formula I, or a pharmaceutically acceptable salt thereof, according to claim 1, comprising:
i) reacting an acid of the formula II, 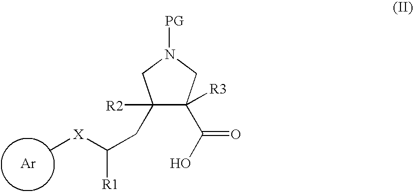
wherein PG is a protecting group, with diphenylphosphorus azide, in the presence of a base, a tri-lower alkylsilyl ethanol to give the corresponding protected amino compound of the formula III, 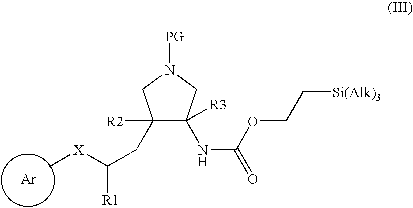
wherein Alk is C₁₋₄-alkyl;
ii) subsequent removing the tri-lower alkylsilylethoxy group to give an amino compound of the formula IV, 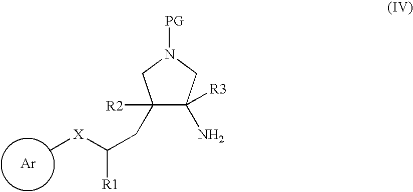
iii) reacting a compound of the formula (IV) with a compound of the formula V,
R5-Y-Z (V)
wherein Z is a leaving group to obtain a compound of the formula VI 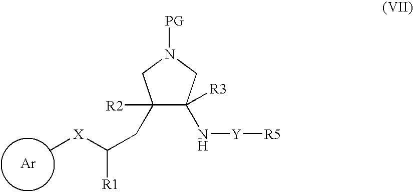
and
iv) removing the protecting group PG to obtain a corresponding compound of the formula I.

24. A process for the manufacture of a compound of the formula I, or a pharmaceutically acceptable salt thereof, wherein R2 is hydroxy, according to claim 1, comprising:
i) oxidizing a compound of the formula VIII, 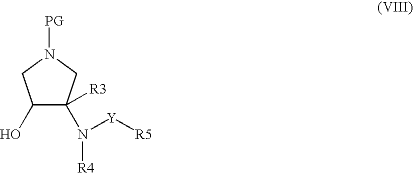
wherein PG is a protecting group, to obtain a compound of formula IX 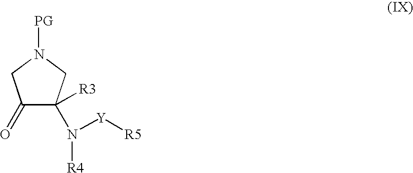
wherein;
ii) reacting the compound of formula IX with a metallo reagent of the formula X,
Ar—X—CHR¹—CH₂—Mg-Hal (X)
wherein Hal is halo, to obtain, upon removal of the protecting group PG, the corresponding compound of the formula I.

25-26. (canceled)
